(19) United States
(12) Patent Application Publication
(10) Pub. No.: US 2023/0366873 A1
(43) Pub. Date: Nov. 16, 2023

(54) SYSTEM AND METHOD TO MEASURE CAR-T CELL QUALITY

(71) Applicant: Carnegie Mellon University, Pittsburgh, PA (US)

(72) Inventors: Marc Peralte Dandin, Pittsburgh, PA (US); Ching-Yi Lin, Pittsburgh, PA (US)

(73) Assignee: Carnegie Mellon University, Pittsburgh, PA (US)

(21) Appl. No.: 18/316,200

(22) Filed: May 11, 2023

Related U.S. Application Data
(60) Provisional application No. 63/340,511, filed on May 11, 2022.

(51) Int. Cl. G01N 33/487; G01N 33/50

(52) U.S. Cl. CPC G01N 33/48735; G01N 33/502; G01N 33/505

(57) ABSTRACT

A system and method utilize capacitance sensor data to identify cell events with single-cell resolution. The method identifies patterns in the sensor data related to events such as mitosis, migration-in to the sensor field, and migration-out. The system may include a processor co-located with the sensor to perform the pattern recognition. Further, microfluidic channels can be provided to direct cells to the sensors.

### CROSS-REFERENCE TO RELATED APPLICATIONS

This application claims the benefit under 35 U.S.C. § 119 of U.S. Provisional Application Serial No. 63/340,511, filed on May 11, 2022, which is incorporated herein by reference.

### STATEMENT REGARDING FEDERALLY SPONSORED RESEARCH

Not applicable.

### BACKGROUND OF THE INVENTION

The present disclosure generally relates to cellular assays. More specifically, the disclosure relates to a system and method to monitor individual cell events to improve the performance of a cellular assay. Cellular culture assays are ubiquitous in biology. Cellular assays can be used to efficiently quantify biocompatibility, cytotoxicity, biological activity, and biochemical mechanisms. The disadvantages of typical assay techniques include limited throughput from complicated fixation processes and the lack of an ability to obtain biologically relevant data in real-time. In addition, with existing assay techniques, it is hard to monitor cell culture precisely and efficiently. As a result, single-cell resolution and high throughput methods are being pursued as alternatives.

Capacitive sensing is a potential alternative to achieve both single-cell resolution and high throughput. For example, cell proliferation has been measured from vertical electrodes, charge-based capacitive measurements (CBCM). Despite their promise, these capacitance sensing methods are best configured for long-term monitoring of cell cultures and thus they lack the ability to monitor life-cycle events at the single-cell level.

Therefore, it would be advantageous to develop a microsystems-based cell assay technique that produces single-cell resolution, permitting identification of cellular events in real-time.

### BRIEF SUMMARY

According to embodiments of the present disclosure is a system and technique utilizing capacitive sensing to identify and classify various cell events at the single-cell level. In one embodiment, the system comprises a cell culture well for housing cells under study and a CMOS-integrated capacitance sensor array for measuring cell proliferation in real-time. The device forms a lab-on-CMOS microsystem capable of autonomously monitoring cell cultures over long periods of time.

The capacitive sensors are combined with a temporal pattern recognition process to obtain relevant biological data from the sensor data. The capacitive sensors are sensitive to single-cell operations, such as mitosis or migration. Cell mitosis and migration are particularly important in cancer cell characterization, such as those involving chimeric antigen receptor T-cell (CAR T-cells). CAR T-cells are cells collected from a patient and re-engineered to assist the patient’s immune system in attacking cancer cells. Cellular assays are vital in assessing the viability and functionality of these cells collected from a patient.

The system and method of the present disclosure bridges the gap between high-resolution single-cell measurements and capacitive sensing by extracting various signal micro-patterns pertaining to single cells from measured capacitance data. Specific cell behaviors, such as mitosis or migration, are modeled as spatio-temporal events. Cell behavior can be associated with events in the data using pattern recognition.

### BRIEF DESCRIPTION OF THE SEVERAL VIEWS OF THE DRAWINGS

FIG. 1 is a schematic of a sensor according to one embodiment.

FIG. 2 is a diagram of a sensing electrode.

FIGS. 3A-3C are graphs depicted capacitance over a time series related to different cellular events.

FIG. 4A is a sensor according to an alternative embodiment.

FIG. 4B is a sensor according to yet another embodiment.

FIG. 4C shows a microfluidic network associated with a plurality of sensors.

FIG. 5 is a schematic of a capacitance sensor.

FIGS. 6A-6C show graphs of sensor data in raw, compressed, and slope merged formats.

FIGS. 7A-7B show graphs with pattern recognition.

FIG. 8 depicts search success and failures.

FIGS. 9A-9B aregraphs of features, showing mitosis and migration events.

FIG. 10 is a graph depicting measured capacitance over time.

FIG. 11 are histograms comparing different capacitance measurements.

### DETAILED DESCRIPTION

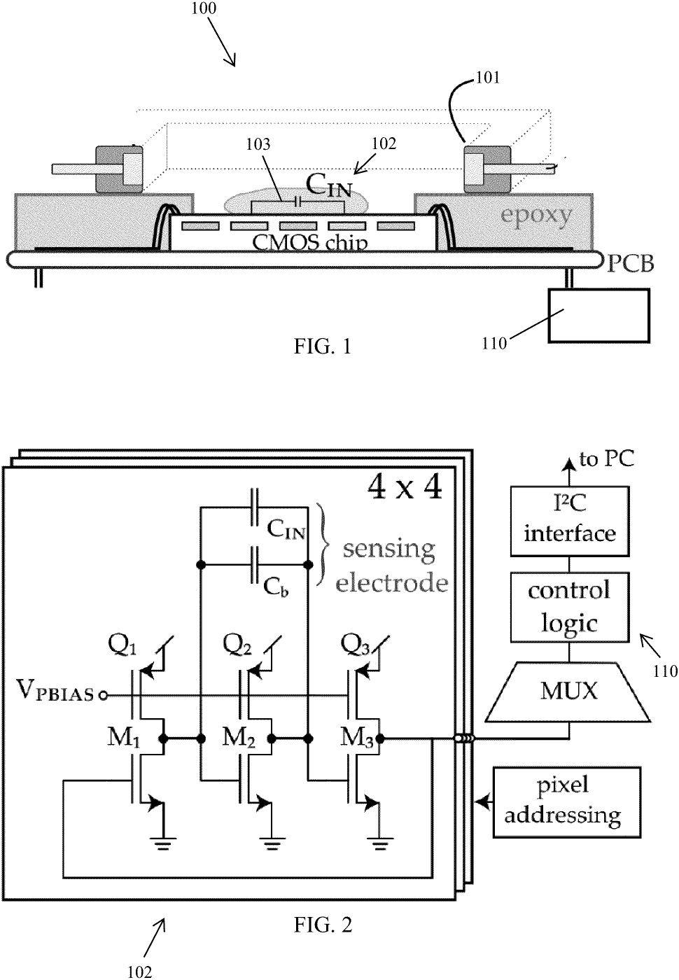

According to embodiments of the disclosure is a system 100 and method for sensing and classifying cell behavior in a cellular assay with single-cell resolution. FIG. 1 shows the system 100 comprising a culture well 101 and a sensor 102. The sensor 102 shown in FIG. 1 is a complementary metal-oxide semiconductor (CMOS) die wire bonded to a printed circuit board (PCB). FIG. 2 is a detailed view of the circuit architecture of the sensor 102. As depicted in FIG. 2, the sensor 102 is an application-specific integrated circuit (ASIC) fabricated in a 0.35 µm commercial CMOS process. The ASIC includes capacitance biosensors 102 in the form of passivated interdigitated electrodes 103 as well as control and data dispatch circuits 110. The biosensors 102 are configured to monitor changes in interfacial capacitance in a region directly on top of the electrodes 103.

The sensors 102 measure the input capacitance C_{IN} as it changes during cell life-cycle events. The measurement also includes a deterministic baseline capacitance C_{b}, which represents the effective stray capacitance at the electrode 103. The measured capacitance is mapped to the frequency of a three-stage NMOS ring-oscillator, and the oscillator’s output signal is fed to data processing circuits 110 that estimate its frequency by counting the number of rising edges that occur during the measurement period. Alternatively, the sensor 102 can be based on a charge-based capacitance architecture.

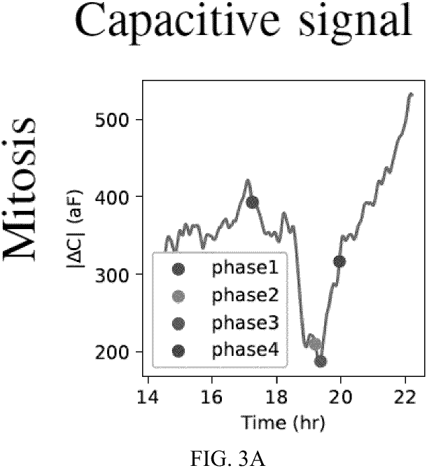

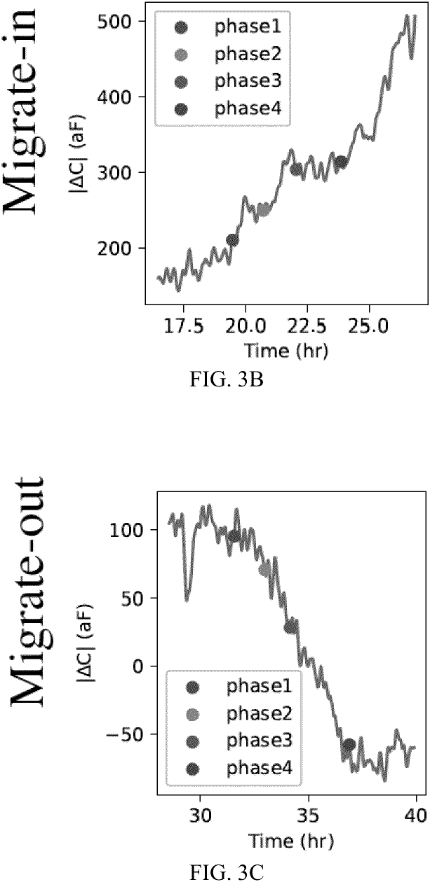

Three representative cellular events are shown in FIGS. 3A-3C, with sensed capacitance shown over a period of 72 hours. A mitosis event is a cell division process characterized by (FIG. 3A): a detachment phase (phase 2), a division phase (phase 3), and an adhesion phase (phase 4). This process is manifested as a V-shape pattern in the capacitive sensor signal, with phases 2-3 located near the trough of the V-shape pattern. Migration-in/out events are movements towards and away from the electrodes 103, which generate a rising or falling slope corresponding to a change in the cell’s coverage of the electrode 103.

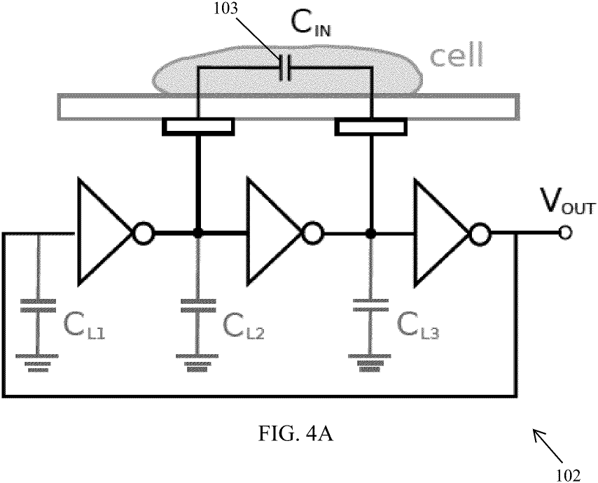

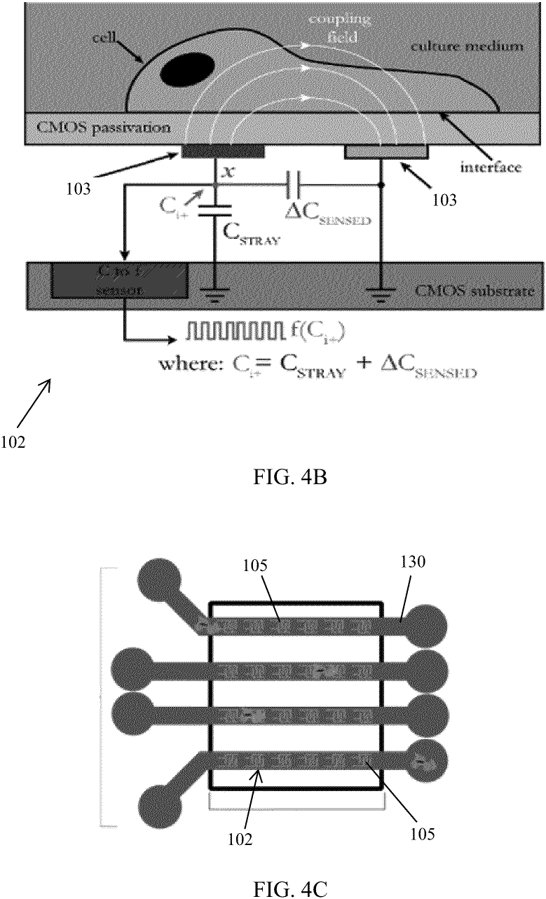

In the embodiment used to produce the capacitance signals in FIGS. 3A-3C, the sensor 102 comprises a pair of conductors 105 separated by an insulator, thus forming a sensing electrode 103. FIG. 4A is a schematic of design of this sensor 102 and is a 3-stage oscillator with cell capacitance C_{IN}, which generates output with the frequency as a function of C_{IN}. When the region between the conductors 105 is perturbed by a cell, there is a change in the capacitance sensed. This change can be modeled according to: C = εA/d ∝ ε, where ε is the permittivity of the medium, A is the area of the parallel plates formed by the two conductors 105, and d is the distance between two parallel plates. With the observed higher permittivity of the cultured cell compared to the medium, the measured signal can be used to characterize the cell’s activity at the electrode site in real-time.

Although prior works show an ability to monitor a cell culture assay, these works focus on the macro-scale properties and cannot provide single-cell resolution. In contrast, the sensor 102 of the present system 100 observes the capacitance change from different cell behaviors. Further, an identification and classification framework captures these micro-scale cell properties.

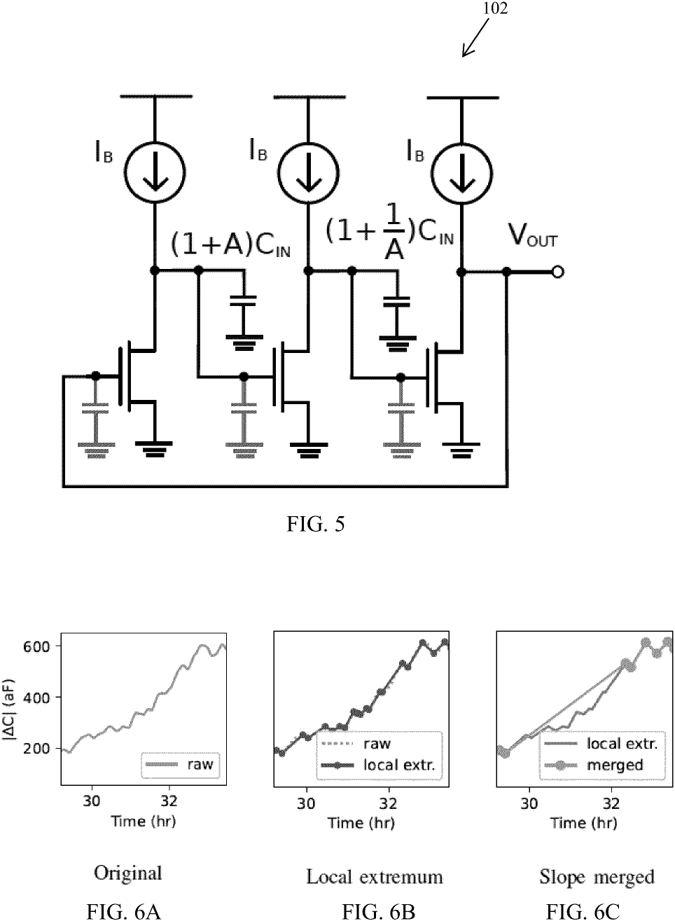

The sensor 102 shown in FIG. 4A is based on a capacitance-to-frequency transduction mechanism. The transduction of cell life-cycle events is achieved by connecting an interdigitated electrode 103 (with C_{IN}) to two internal nodes of a three-stage ring oscillator. FIG. 5 shows the implementation: the oscillator is implemented with pseudo-inverters with bias current I_{B} and (parasitic) capacitance C_{L,i} at each stage. Assuming constant I_{B} and NMOS turn-on current I_{NMOS}, the oscillator period is proportional to total load capacitance C_{L} = C_{L1} + C_{L2} + C_{L3} and can be expressed as T = kC_{L} with some constant k.

Considering the electrodes 103, the overlying cell introduces extra capacitance and thus reduces the oscillator frequency. From Miller’s effect, this floating C_{IN} is equivalent to a (1+A) C_{IN} and a (1+ A⁻¹) C_{IN} at each node with small-signal gain A (shown in FIG. 5) and results in the oscillator frequency:

$$f = \frac{1}{T} = \frac{1}{k} \times \frac{1}{{C}_{L} + \left(\left(1 + A\right) {C}_{I N} + \left(1 + {A}^{- 1}\right) {C}_{I N}\right)} \qquad \text{(1)}$$

The linearity between f and C_{IN} can be achieved with the assumption C_{L,i} >> (2 + A + 1/A)C_{IN} and approximated with f = -α C_{IN} + ƒ₀. Its parameters slope α and intercept ƒ₀ can be obtained by fitting the simulation data into a linear function.

FIG. 4B shows yet another alternative embodiment of the system 100 comprising a high-density 2D array of capacitance sensors 102 and a microfluidic network 130 overlying the array of sensors 102. The sensor 102 comprises an insulated electrode pair 103 and underlying control and processing circuits 110. The circuits 110 are configured to monitor changes in interfacial capacitance in a region directly on top of the electrodes 103. Specifically, the circuits 110 measure the input capacitance as it changes when a cell is present at the interface. The effective input capacitance Cᵢ₊ at the node x is mapped to a frequency f(Cᵢ₊), where Cᵢ₊ is the sum of ΔC_{SENSED} and the deterministic parasitic capacitance (C_{STRAY}) at the node x. In a cell assay using this system 100, when a cell gets close to the interdigitated electrode 103, ΔC_{SENSED} changes from its baseline value and this change is mapped to the frequency to the test signal.

Cellular chemotaxis is a crucial step in invoking an effective immune response and a hallmark of immune cell activation. Migration can be used as measurement of CAR-T cell activation. Moreover, the migration event can facilitate CAR-T cell subpopulation segregation, thereby enabling collection for downstream characterization. The microfluidic CMOS sensor system 100 shown in FIG. 4B can be functionalized with a chemokine gradient to facilitate measurement of the chemotaxis process (FIG. 4C). Cells isolated via this approach could be analyzed for the abundance of different cytokines (or other genes associated with activity) by Multiplex Immunoassay and Luminex. The system 100 can be used with any number of chemokines, allowing customization of the selection property for the isolated cell populations.

Further, the system 100 can be used to measure changes in migration speed and directionality which may provide an additional quality assessment measurement. Algorithms can be designed to predict cell behavior such as viability and proliferation from the capacitance measurement alone, so multiple devices can be maintained inside a cell culture incubator and a bulky microscope and microscopic live cell culture system is not needed. Previous works have shown that changes in sensed capacitance across time are highly correlated with cell proliferation and motility. Identifying key features from the signals from the sensor 102 can allow recognition of cellular events.

### Pattern Recognition

Once capacitance data is obtained from the sensor 102, a pattern recognition process is used to identify cellular events and is based on the sensed capacitance as a function of time. More specifically, the process of identifying temporal features in a time interval utilizes a representation technique and a similarity metric to quantify the likelihood of features associated with an event. The representation technique maps data from a high dimensional domain into another space with lower complexity or more straightforward representation and the consequent similarity metric defines the closeness of two data points (or subsequences) in the new space with prior knowledge. The process builds on the advantages of linear approximation and with some modifications that improve the computational complexity.

The pattern recognition process can performed on the circuit 110 co-located with the sensor 102 or it may be a separate module. The module may comprise a controller, a microcomputer, a microprocessor, a microcontroller, an application specific integrated circuit, a programmable logic array, a logic device, an arithmetic logic unit, a digital signal processor, or another data processor and supporting electronic hardware and software.

Similarity is a metric that can be hard to evaluate. Due to the difficultly in determining similarity, some prior works instead defined a distance metric and the inverse relation between them (short distance implies high similarity). This simplified distance metric does not always produce accurate results. The method of the present disclosure uses a customized distance metric and symmetry degree as the similarity metric, improving accuracy.

In the following example embodiment, the types of cell behaviors that will be recognized are described as follows:
(1) Migrate-in event - Occurs when the cell moves onto the sensor electrode 103. This event generates a rising slope in the signal, as shown in FIG. 3B.
(2) Migrate-out event - This event is opposite to the migrate-in event and is represented as a falling slope in the signal, as shown in FIG. 3C.
(3) Mitosis event - This event comprises two or more phases. For example, cell detachment and attachment are shown as a falling slope followed by a rising slope, forming a V-shaped pattern in the signal, as shown in FIG. 3A.

To identify these three temporal patterns, the classification method comprises the following steps: At step 201, a piecewise linear approximation is performed upon the signal. At step 202, a conjugate search finds the conjugate slope and forms a slope pair, where the slope pair comprises the slope and its conjugate. And at step 203, the cell behavior classification based on the symmetry within the slope pair is determined. This symmetry is used as the likelihood of cell mitosis events.

### Linear Approximation

During step 201, a pre-processing routine converts a time series of capacitance measurements received from the sensors 102 into an interpretable representation. In this embodiment, a piece-wise linear function is used as the representation, which reduces an N-point time series{(tᵢ, yᵢ)}, for i = 1, 2, ..., Ninto (tⱼ, yⱼ), for j = 1, 2, ..., M, where Mis a number of linear segments, where M < N, and the value between any two adjacent points is interpolated.

To reduce the computation required to find a new approximated value, the series is simplified by making each data point in the approximation sequence a local extreme in the original sequence. Equivalently, this linear approximation finds a subset of the indices {i|yᵢ = max{yᵢ - 1, yᵢ, yᵢ + 1} or yᵢ = min{yᵢ - 1, yᵢ, yᵢ + 1}}.

Although the operation compresses the sequence greatly, as shown in FIGS. 6A-6B, it might contain saw-tooth patterns if the trend is not clear. Thus, another operation, slope merge, is performed. During the slope merge operation, short slopes are lumped into slopes of bigger segments, effectively merging a plurality of slopes. This step removes the short slopes and further increases the compression ratio. FIG. 6C shows the series after the slope merge operation.

More specifically, given a tolerance parameter k, if Vj = i + 1, i + 2, ..., i + m - 1 satisfies g(i,j) ∈ [1/k * g(i, i+m), k * g(i, i+m)] and g(j, i+m) ∈ [1/k * g(i, i+m), k * g(i, i+m)], with gradient function g(i, j) - (yⱼ - yᵢ)/(χⱼ - χᵢ), then {tᵢ₊₁, tᵢ₊₂, ..., tᵢ₊ₘ₋₁} is removed from the approximated series.

The linear approximation converts the signals from the sensor 102 into a more compact representation but introduces some error. The result is quantified in terms of a compression ratio and a median error. The compression ratio is defined as the ratio of the number of data point between the piecewise linear function and the raw signal.

### Conjugate Search

From the signal as a piece-wise linear function, step 202 is used to find the slope pair and determine if this signal represents a mitosis event. This process step pairs the reference segment to another segment, which is called the conjugate, and this conjugate is hypothesized as the segment with the most horizontal connection to the reference segment. To quantify the degree of horizontal connectivity, the process step starts from defining the conjugate of a point first and extends the concept to the segment.

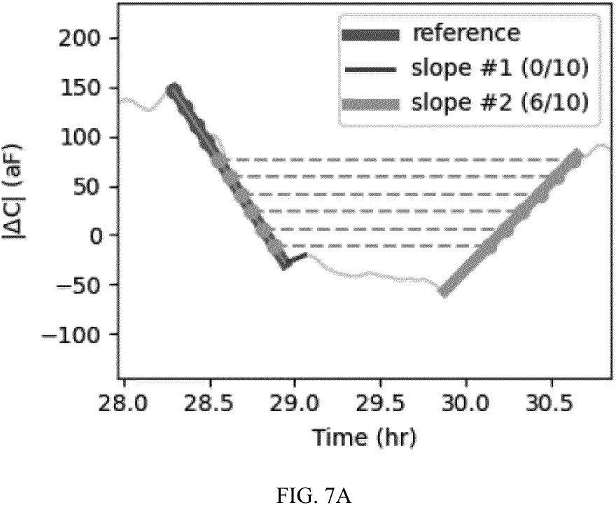

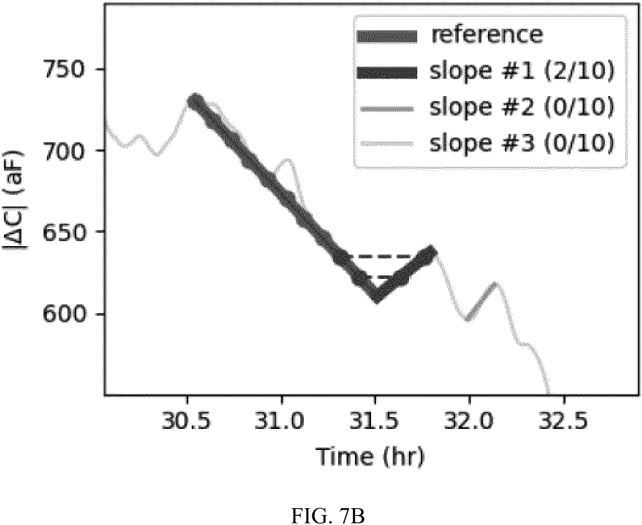

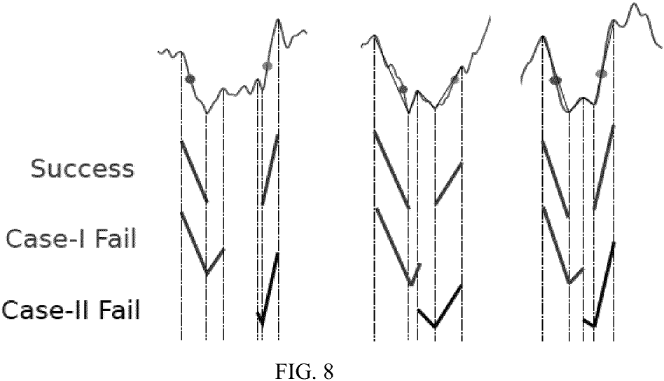

The conjugate point is defined as a point connected to the reference point by a horizontal line, as shown in each pair of terminal points in the dashed lines of FIGS. 7A-7B. This pointwise relationship can be extended to segments. Here the term “segment” is used because this process step is not limited to a straight slope. The conjugate segment is defined as a segment with the highest horizontal connectivity, or the most points that connect to corresponding points on the slope. This connectivity is estimated from the empirical conjugate point ratio from some uniformly-sampled points. The mitosis event in FIG. 7A illustrates an example where the reference slope (left side) has 10 sampled points and the second slope (right side) contains six conjugate points. This implies that if the reference slope is the falling slope of the mitosis event, this slope is most likely its counterpart in the event. FIG. 7B shows a migrate-out event and does not display the same V-shaped pattern as the mitosis event.

A successful result of step 202 can demonstrate the following parameters: (1) the first phase (falling-slope) of each mitosis event finds its second phase (rising-slope); and (2) each falling slope is not excluded from an optional filter in the conjugate search. Although the conjugate search during step 202 can find the conjugate for any segment, some slope pairs are not reasonable and unlikely to be a meaningful event. For example, a slope pair with a long time gap or a conjugate with a very small amplitude is usually not part of a mitosis event. To prevent the redundant pairs, in one alternative embodiment, the process uses two filters before the search process to select proper falling slopes. In the first, the filter only applies on the significant segments obtained from adjacent sample points. In the second filter, the region of conjugate search is limited to a finite time range. A slope pair with long time interval between each segment usually does not represent the same behavior. A time limit thus is set to conjugate search process.

In addition, the conjugate search process in step 202 can emphasize sensitivity more than specificity. Stated differently, finding more false positive slopes can be more beneficial than missing a potential conjugate since the false positive slopes can be filtered out from the mitosis-migration classification during step 203, but missed slope pairs will not be classified. Failures to find a conjugate can result from finding the wrong conjugate or not identifying the falling slope as a significant slope. FIG. 8 shows the various errors. The top lines represent successful conjugate searches. The middle represents an unsuccessful search, where the wrong conjugate was identified. And the bottom lines represent an unsuccessful search where the falling-slope is not a significant slope. Although migration events (migrate-in, migrate-out) do not have a conjugate pair in their pattern, they still suffer from second failure if the slope is not recognized as a significant slope (too flat or with too short of a time interval) and masked out from the pre-filter before conjugate search.

### Classification From Degree of Symmetry

Each slope pair found in step 202 can be characterized by the gradient s of each slope of the pair and the y-value difference Δy of the first and second slope. Since a symmetric V-shaped pattern implies that the gradient s of the first and second slope y-value difference is equal for each slope, the difference of these attributes can be used as a V-shape likelihood score.

Feature design - The observation above inspires the design of the features in step 203, where the difference in the log of each slope and the difference in the log of each y-value difference is determined. A log-difference operator makes the classification insensitive to amplitude scaling. With the feature design based on the symmetry, the norm of the feature vector indicates the degree of symmetry, which is zero for the perfectly symmetric slope pair. Specifically, if each slope pair can be characterized as ((s₀, Δy₀), (s₁, Δy₁)), then Δlog(s) = log(s₁) - log(-s₀) and Δlog(Δy) = log(y₁) - log(y₀). The log-difference operator Δlog(a,b) = log(|a|) - log(|b|) is insensitive to amplitude scaling, that is, Δlog(a,b = Δlog(ka, kb).

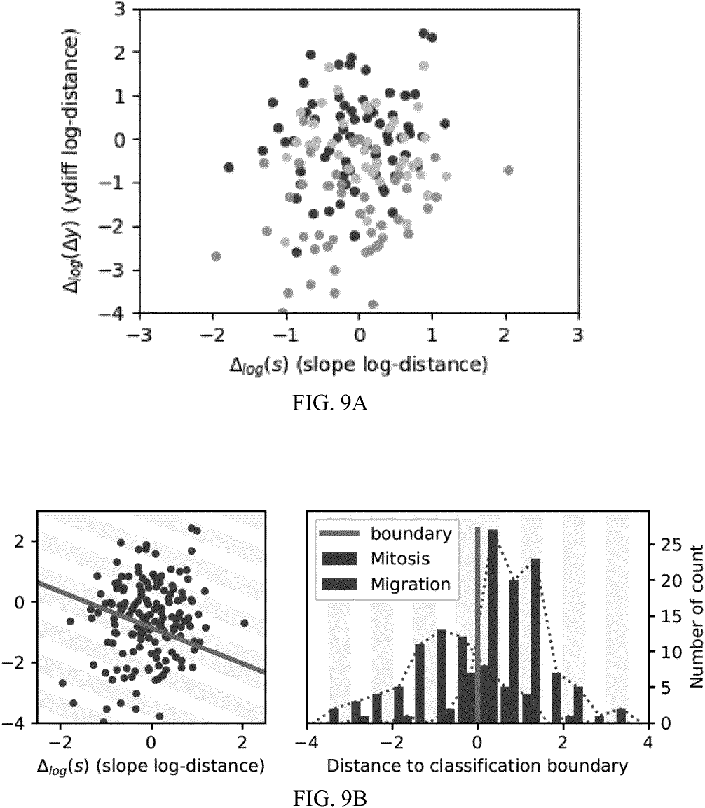

FIG. 9A visualizes the features from two cell behaviors, mitosis (top) and migration-out (bottom). This scatter plot shows a mitosis cluster centered at the original point and a migration cluster at the third quadrant. The smaller vector length of mitosis events validates the fact that these events are more symmetric than the migration events. The negativity of the migration features is because of the lack of a rising slope during the migration-out event. FIG. 9B is another set of graphs showing the distinction between mitosis and migration events.

Linear boundary from SVM - The boundary can be searched from support vector machine (SVM). The boundary is a measure that quantitatively delineates one biophysical cue from another. In one embodiment, the C-Support Vector Classification package from scikit-learn is used with linear kernel and default parameters except for balanced class weight. The balanced class weight option is set to reduce the bias from class distribution of labelling by equalizing the frequency of both classes.

Once a pattern is found for a slope pair using the method of the present disclosure, the pattern recognition steps can be used on other data sets to look for similar patterns. For example, the pattern recognition steps can be utilized on a training set where cellular events are confirmed via visual observation. Once the pattern is detected in this training set, the process can be extended to more data sets.

Using the system 100 of the present disclosure, six experiments were performed to study the effects of tumor treating electrical fields (TTFields) on human breast cancer cells obtained from a commercially available cell line. In each experiment, 4 mL of a cell solution was added to the microsystem’s culture well. The starting cell density was ~75,000/mL, resulting in approximately 300,000 cells in the media.

The microsystem was placed in the incubator for 72 hours. Of the six experiments, three were performed without TTField electrodes being energized and thus served as a control. The remaining three experiments were conducted with the TTField electrodes energized and commutated.

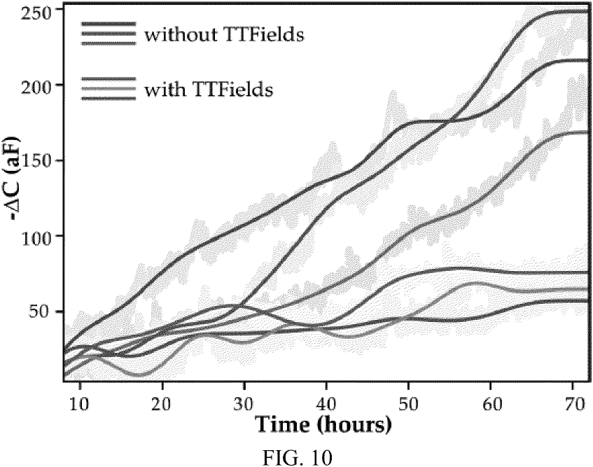

The results of the six experiments are shown in FIG. 10. The shaded traces are the measured sensor data averaged for 16 electrodes for each experiment. The solid traces represent a Savitsky-Golay smoothing filter which preserves the trend of the averaged data. The results shown in FIG. 10 reveal that without TTFields the measured change in capacitance reported by the sensor 102 continually increased, a characteristic indicative of unimpeded cell growth. Conversely, in cases where the TTField electrodes were energized and commutated, the measured change in capacitance exhibited a much slower growth as evidenced by an average 100 aF difference in the endpoints of the experiments as compared to the control. As such, these results confirm that the anti-cancer effects of TTFields can be monitored in real time using a label-free capacitance sensor 102 configured for measuring cell proliferation.

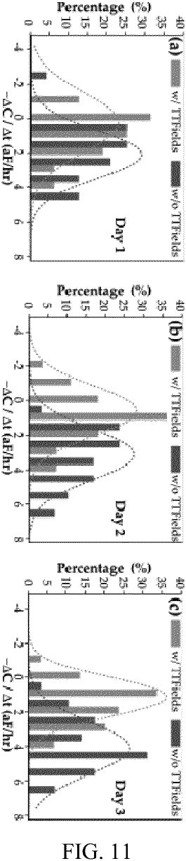

Further, the slopes of consecutive and overlapping segments of the time series data can be determined to infer the cell population’s growth over short periods of time. For each of the six experiments, the slopes (-ΔC/Δt) were estimated using linear regressions on data from a set of sliding windows (10 hours with 75% overlap) extending over the entirety of the 72-hour period. FIG. 11 is a slope histogram metric computed for the 3-day period. By day two there was a clear shift between the mean slope value for cell populations undergoing TTField exposure and that of cell populations that remained unexposed to TTFields. Particularly, without TTFields, the slopes tended to be higher, indicating high rates of cell growth, and conversely, with TTFields, the slopes were on average smaller, indicating impeded cell growth.

When used in this specification and claims, the terms “comprises” and “comprising” and variations thereof mean that the specified features, steps, or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure. Although certain example embodiments of the invention have been described, the scope of the appended claims is not intended to be limited solely to these embodiments. The claims are to be construed literally, purposively, and/or to encompass equivalents.

What is claimed is:

1. A device comprising:
a plurality of capacitance sensors integrated on a complementary metal-oxide semiconductor; and
a microfluidic network comprising one or more channels disposed on the metal-oxide semiconductor,
wherein the one or more channels overlap with sensing fields of the plurality of capacitance sensors;
wherein a capacitance sensor from the plurality of capacitance sensors is configured to measure a capacitance associated with a biophysical cue of a cell located in one of the one or more channels.

2. The device of claim 1, wherein the biophysical cue is a migration of the cell within the sensing field.

3. The device of claim 1, wherein the biophysical cue is a mitosis of the cell.

4. The device of claim 1, wherein the cell is a chimeric antigen receptor T cell.

5. The device of claim 1, wherein the cell is an immune system cell.

6. The device of claim 1, further comprising:
a circuit that receives data from the plurality of capacitance sensors.

7. The device of claim 6, wherein the circuit is integrated with the complementary metal-oxide semiconductor.

8. The device of claim 6, wherein the circuit is adapted to recognize a pattern in a change of the capacitance over a period of time.

9. A device, comprising:
a complementary metal-oxide semiconductor chip;
a plurality of capacitance sensors integrated on the chip;
a processor communicatively coupled to the plurality of capacitance sensors, wherein the processor is configured to classify two or more biophysical cues of a cell measured by a capacitance sensor from the plurality of capacitance sensors.

10. The device of claim 9, wherein the two or more biophysical cues include a biophysical cue selected from the group consisting of: mitosis, migration towards a predetermined location on the chip, and migration away from the predetermined location on the chip.

11. The device of claim 9, wherein the cell is a chimeric antigen receptor T cell.

12. The device of claim 9, wherein the cell is an immune system cell.

13. The device of claim 9, wherein the processor is integrated with the chip.

14. The device of claim 9, wherein the processor is co-located with the chip.

15. A method of identifying cellular events in a cellular assay comprising:
obtaining a plurality of capacitance measurements over a period of time from a sensor in contact with a cell;
identifying a trend in the plurality of capacitance measurements;
identifying a conjugate trend in the plurality of capacitance measurements;
calculating a degree of symmetry between the trend and the conjugate trend; and
associating a cellular event with the degree of symmetry.

16. The method of claim 15, wherein the cell is a chimeric antigen receptor T cell.

17. The method of claim 15, wherein the cellular event is selected from the group consisting of mitosis, migration into contact with the sensor, and migration out of contact with the sensor.

18. The method of claim 15, wherein identifying a trend in the plurality of capacitance measurements comprises:
identifying a rising or falling slope in a dataset comprising capacitance over time.

19. The method of claim 15, wherein calculating a degree of symmetry between the trend and the conjugate trend comprises:
identifying a number of points located on the trend that connect to corresponding points on the conjugate trend.

20. The method of claim 15 further comprising:
removing the trend and the conjugate trend if a large time gap exits between the trend and the conjugate trend.
